(19) United States
(12) Patent Application Publication
(10) Pub. No.: US 2024/0029827 A1
(43) Pub. Date: Jan. 25, 2024

(54) METHOD FOR DETERMINING THE PATHOGENICITY/BENIGNITY OF A GENOMIC VARIANT IN CONNECTION WITH A GIVEN DISEASE

(71) Applicant: Engenome S.R.L., Pavia (IT)

(72) Inventors: Ivan Limongelli, Pavia (IT); Giovanna Nicora, Pavia (IT)

(21) Appl. No.: 18/040,604

(22) PCT Filed: Jul. 28, 2021
(86) PCT No.: PCT/IB2021/056870

§ 371 Date: Feb. 3, 2023

(30) Foreign Application Priority Data
Aug. 4, 2020 (IT) 102020000019180

(51) Int. Cl. G16B 20/20; G16B 40/20; G16H 50/50

(52) U.S. Cl. CPC G16B 20/20; G16B 40/20; G16H 50/50

(57) ABSTRACT

A method is for determining the pathogenicity/benignity of a genomic variant in connection with a given disease includes accessing genomic data in a list of the patient's genomic variants and for each variant detected, verifying whether or not the variant meets each predefined pathogenicity/benignity criteria. Each of such pathogenicity/benignity criterion is a proposition, which can be true or false, related to the variant for a previously known condition or a patient-specific condition. Input information is prepared for a trained algorithm using artificial intelligence and/or machine learning. The input information includes information related to the pathogenicity/benignity criteria associated with the level of evidence met by the variant. The input information is processed by the trained algorithm, to obtain an output information representative of the pathogenicity/benignity of each variant. The algorithm is trained in a preliminary step of training.

### TECHNOLOGICAL BACKGROUND OF THE INVENTION

### Field of Application

The present invention relates to a predictive prognosis method regarding the pathogenicity/benignity of a genomic variant in connection to a given disease.

Therefore, the general technical field of the present invention is that of predictive methods, performed by electronic computation, used in the context of genomics and/or medical genetic research to support predictive prognoses.

### Description of the Prior Art

In the field of medical genetics, the objective of diagnostic tests which analyze a patient's DNA is to find possible mutations, i.e., “variants” which can explain the onset of a disease. These variants are named “pathogenic”, while all other variants which do not cause a pathology but depend on interpersonal differences are named “benign”.

The process of identifying pathogenic variants is named “interpretation of the variants”.

Following a sequencing analysis, thousands of variants may be found for each patient, of which few are actually pathogenic.

Therefore, there is a strong need for computational tools and/or automatic tools to support the interpretation of variants, which make it possible to analyze the large amount of data generated by sequencing and to obtain prognostic results as quickly and reliably as possible.

For this reason, several software programs or modules have recently been developed and used (hereafter named “tools”, according to a widely used terminology, for the sake of brevity) which can be generally classified according to the following groups and differ in purpose as well as for the type of technology used:
(a). In silico prediction tools, which are typically based on data-driven approaches the objective of which is to quantify the impact of the variant on the genic product, e.g., by assessing whether the resulting protein is damaged by the presence of the variant.

This typology comprises well-known tools such as CADD (Rentzsch P., Witten D., Cooper G M., Shendure J., Kircher M. “CADD: predicting the deleteriousness of variants throughout the human genome. Nucleic Acids Res. 2019 Jan. 8; 47(D1):D886-94) and VVP (Flygare S., Hernandez E. J., Phan L., Moore B., Li M., Fejes A., et al. “The VAAST Variant Prioritizer (VVP): ultrafast, easy to use whole genome variant prioritization tool”. BMC Bioinformatics. 2018 Feb. 20; 19(1):57.).

However, this type of tools suffers from the drawback of not guaranteeing an accurate classification of the variants into pathogenic/benign, because a protein may be able to tolerate many damaging mutations (e.g., consider the technical publications: Richards S., Aziz N., Bale S., Bick D., Das S., Gastier-Foster J., et al. “Standards and guidelines for the interpretation of sequence variants: a joint consensus recommendation of the American College of Medical Genetics and Genomics and the Association for Molecular Pathology”. Genet. Med. Off. J. Am. Coll. Med. Genet. 2015 May; 17(5):405-24; and also: Niroula A., Vihinen M. “How good are pathogenicity predictors in detecting benign variants?” PLOS Comput. Biol. 2019 Feb. 11; 15(2):e1006481).
(b). Variant pathogenicity prediction tools, which are also based on data-driven approaches, and often applying machine learning technologies, which involve training to classify variants into pathogenic or benign.

Examples of such a type of tools are ClinPred (Alirezaie N., Kernohan K. D., Hartley T., Majewski J., Hocking T. D. ClinPred: “Prediction Tool to Identify Disease-Relevant Nonsynonymous Single-Nucleotide Variants”. Am. J. Hum. Genet. 2018 Oct. 4; 103(4):474-83.) or LEAP (Lai C., Zimmer A. D., O'Connor R., et al. LEAP: “Using machine learning to support variant classification in a clinical setting”. Hum. Mutat. 2020; 41(6):1079-1090. doi:10.1002/humu.24011), or again as described in:
Hu Z., Yu C., Furutsuki M., et al. “VIPdb, a genetic Variant Impact Predictor Database”. Hum. Mutat. 2019; 40(9)1202-1214. doi:10.1002/humu.23858.

However, this type of tools suffers from the drawback of not guaranteeing a standardized classification and offers results which are largely disconnected from official guidelines, provided by medical/clinical institutions, which are typically used by physicians/geneticists, because they are considered essential to guarantee uniformity and accuracy to the interpretation of variants.
(c). In light of the above, other types of pathogenicity prediction tools were developed which implement one or more of the above guidelines for variant interpretation.

Examples of such guidelines comprise the ones developed by the American geneticist associations ACMG/AMP (Cf. in this regard: Richards S., Aziz N., Bale S., Bick D., Das S., Gastier-Foster J., et al. “Standards and guidelines for the interpretation of sequence variants: a joint consensus recommendation of the American College of Medical Genetics and Genomics and the Association for Molecular Pathology”. Genet. Med. Off. J. Am. Coll. Med. Genet. 2015 May; 17(5):405-24.)

The ACMG/AMP guidelines provide a set of rules for combining available variant information and patient features to classify each variant into a class. The ACMG/AMP guidelines provide a classification into one of the following five classes: Pathogenic, Likely pathogenic, Benign, Likely benign, VUS (i.e., uncertain).

The interpretation process according to ACMG/AMP guidelines is divided into two parts.

(i). Determining how many criteria for pathogenicity and benignity each variant meets. These criteria are the features of the variants.

The criteria are divided into different levels of evidence in favor of whether the variant is pathogenic or not. In the example implemented by the ACMG/AMP guidelines, there are seven levels of evidence: “pathogenic very strong”, “pathogenic strong”, “pathogenic moderate”, “pathogenic supporting”, “benign stand-alone”, “benign strong”, and “benign supporting”.

(ii). Once all the criteria the variant meets are associated with it, a set of IF-THEN rules combines the number of criteria into the various levels of evidence to determine the final classification. However, such IF-THEN criteria remain at a rather general level and prescribe a minimum number of criteria which must be met for a variant to be classified as benign or pathogenic. Because of this, many variants, i.e., all those variants which do not meet the minimum number of criteria needed to classify it as benign and which also do not meet the minimum number of criteria needed to classify it as pathogenic, are classified as “uncertain.”

Obviously, the higher the number of variants which a model or tool or guideline classifies as uncertain, the lower the predictive quality and reliability.

In this regard, the tools which simply automate guideline interpretation suffer from the same drawback as the guidelines, i.e., they classify numerous variants as “uncertain” and provide less than satisfactory predictive quality and reliability.

Since their publication in 2015, the ACMG/AMP guidelines have been adopted by most laboratories worldwide to standardize the interpretation process.

Consequently, the tools which implement ACMG/AMP (type (c)) are, in this respect, preferred over specifically data-driven pathogenicity prediction tools (type (b)—which, as noted above, implement non-standardized black-box methodologies) because they follow an official standard.

On the other hand, such tools which implement guidelines, as noted above do not allow to classify the many uncertain variants, which makes them less performing in this respect than data-driven tools.

Thus, the need, increasingly felt in the considered technical field, to have tools for classifying genomic variants into benign or pathogenic which, on the one hand, relate to standard guidelines and, on the other hand, can classify as many variants into benign or pathogenic as possible (minimizing the number of “uncertain” variants) and ultimately improving the effectiveness and predictive accuracy, remains unmet.

### SUMMARY OF THE INVENTION

It is the object of the present invention to provide a method for determining the pathogenicity/benignity of a genomic variant in connection to a given disease which makes it possible to solve, at least in part, the drawbacks described above with reference to the prior art and to respond to the aforesaid needs particularly felt in the considered technical sector. Such an object is achieved by a method according to claim 1.

Further embodiments of such a method are defined in claims 2-28.

### BRIEF DESCRIPTION OF THE DRAWINGS

Further features and advantages of the method according to the invention will be apparent in the following description which illustrates preferred embodiments, given by way of indicative, non-limiting examples, with reference to the accompanying figures, in which:

FIG. 1 shows an embodiment of the method according to the present invention by means of a simplified block chart;

FIG. 2 shows a further embodiment of the method according to the present invention again by means of a simplified block chart;

FIG. 3 shows further steps performed by the method, according to a further embodiment of the method according to the invention by means of another simplified block chart.

### DETAILED DESCRIPTION

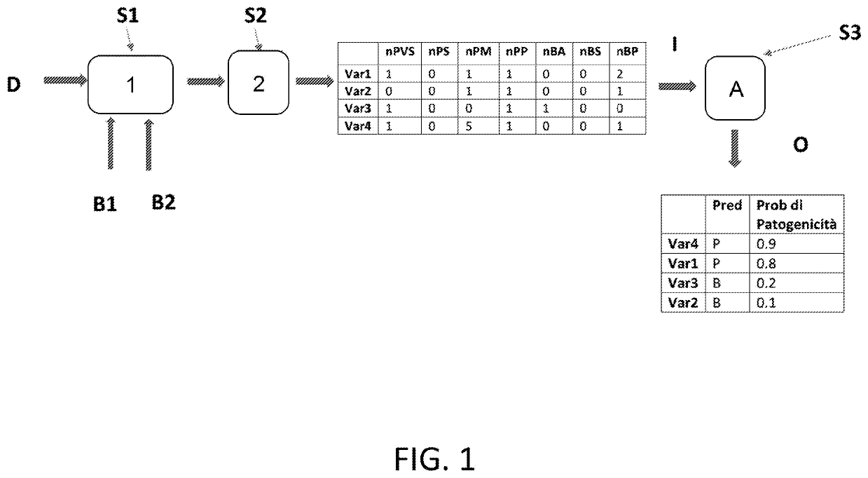

A method for determining the pathogenicity/benignity of a genomic variant in connection with a given disease is described with reference to FIGS. 1-3.

Such a method firstly comprises the steps of accessing genomic data D comprising a list of the patient's genomic variants and then, for each variant detected, verifying (S1), by electronic computing means, whether or not the variant meets each of a plurality of predefined pathogenicity/benignity criteria.

Each of such pathogenicity/benignity criterion is a proposition, which can be true or false, related to the variant, in connection with a first type condition or a second type condition, and wherein at least one of the aforesaid pathogenicity/benignity criteria refers to a first type condition, and at least another one of the pathogenicity/benignity criteria refers to a second type condition.

A “first type condition” comprises a statistical condition and/or a previously known condition.

A “second type condition” comprises a patient-specific condition.

Each pathogenicity/benignity criterion is associated with a level of evidence, indicative of a condition or level of pathogenicity or benignity.

The method then provides preparing (S2), by means of processing by electronic computing means, input information I for a trained algorithm A. Such input information I comprises, for each variant and for each level of evidence, information representing the number of pathogenicity/benignity criteria associated with the level of evidence which are met by the variant.

Finally, the method comprises the steps of processing (S3) the aforesaid input information I by the trained algorithm and of obtaining output information O from the trained algorithm A, wherein the output information O represents the pathogenicity/benignity of each of the genomic variants considered.

The aforementioned trained algorithm is an algorithm which is trained by means of artificial intelligence and/or machine learning techniques (which will also be referred to hereafter as a “machine-learning type algorithm”).

The algorithm A of the machine-learning type used in the present method is trained in a preliminary step of training (S0), based on a training dataset of known cases, providing the algorithm A to be trained with the aforesaid input information calculated for each of the known cases I0, and training the algorithm A based on the knowledge of the pathogenicity/benignity of the respective known cases.

According to an implementation option of the method, the aforesaid output information comprises an estimated probability of pathogenicity of at least one considered genomic variant.

According to another implementation option of the method, the aforesaid output information comprises an estimated probability of pathogenicity of a plurality of genomic variants among the considered genomic variants.

According to another implementation option of the method, the aforesaid output information comprises an estimated probability of pathogenicity of all the genomic variants considered.

According to an embodiment of the method, the output information further comprises, for each genomic variant, a binary result representative of whether the genomic variant is “pathogenic” or “benign”.

Such a result is obtained by comparing the probability of pathogenicity estimated for the genomic variant with a respective threshold, associated with the genomic variant itself.

According to a preferred implementation option of the aforesaid embodiment, the aforesaid respective threshold is an optimized threshold, common for all variants, and determined based on a pre-training.

According to an implementation option, the determination of the optimized threshold is performed in a step of post-processing of the model based on machine learning.

In particular, the pathogenicity decision threshold is shifted from an initial value of 0.5 to a value which optimizes precision, i.e., the percentage of pathogenic variants correctly identified among all those predicted to be pathogenic.

This optimization is important because, as known, the pathogenic variants have a much lower number than the benign ones, and consequently it is important not only to identify the pathogenic variants but to make sure that the number of False Positives (i.e., benign variants predicted as pathogenic) is low, otherwise the list of pathogenic variants that the geneticist must assess could be too large and consequently the effective support to the interpretation is lost.

This procedure is based, for example, on the assessment of the measurement:

$${F}_{β} = \frac{( {β}^{2} + 1 ) \times Precision \times Sensitivity}{{β}^{2} \times Precision Sensitivity}$$

where F₆₂ is a measurement of the performance of a binary classifier (i.e., a classifier in which there are two classes) used in Machine Learning (regarding this, consider e.g., https://en.wikipedia.org/wiki/F1_score) which considers both the capability to correctly classify examples of the “positive” class and the precision with which classification occurs. In the case considered here, the positive class is “pathogenic”.

Precision is the fraction of predicted pathogenic variants which are actually pathogenic, whereas sensitivity is the ability to correctly detect pathogens. Thus, precision and sensitivity are calculated using the following formulae:

Precision=TP/(TP+FP)

Sensitivity=TP/(TP+FN)
wherein:
TP (True Positive)=number of pathogenic variants correctly classified as pathogenic by the algorithm;
FP (False Positive)=number of benign variants incorrectly classified as pathogenic;
FN (False Negative)=number of pathogenic variants incorrectly classified as benign.

These counts vary according to the decision threshold of the algorithm; indeed, the trained machine learning algorithm outputs the probability that a variant is pathogenic.

The final class (benign or pathogenic) is assigned according to this probability; in the “standard” case the two classes are considered “equivalent” and thus when the probability of pathogenicity is >=0.5 then the variant is considered pathogenic.

As this decision threshold varies, the TP, TN, FN, and FP counts change, and it is possible to select an “optimal” classification threshold based on the needs, required by the present application, to be very accurate while maintaining a sensitivity value which is not too low.

In an implementation option, the optimal threshold can then be determined as follows.

The β factor is chosen to assign a higher importance to precision than to sensitivity.

For example, one can empirically choose β=0.35. Such a factor β is thus a “weight” which, according to the formula seen above, makes it possible to favor precision.

At this point, F_{β} is computed at different values of classification thresholds, and the threshold value for which F_{β} is greater is chosen as the optimal (or optimized) threshold.

According to a preferred embodiment of the method, the aforementioned trained machine learning algorithm A is an LR Logistic Regression algorithm.

According to other possible embodiments, the trained algorithm A of the “machine learning” type belongs to a group comprising the following algorithms: Decision Tree, Random Forest, Naive Bayes, Gradient Boosting, Support Vector Machine.

According to an embodiment, the method comprises, before using the aforementioned trained algorithms A, a further preliminary step of training (SO), performed based on the two subsets of the aforementioned training dataset containing data referring to known cases: a first subset (which will also be referred to as the “training set”) is used as the training database and a second subset (which will also be referred to as the “validation set”) is used as the validation database.

According to a particular implementation option, the training dataset is instead divided into three subsets comprising, in addition to the aforementioned first subset and second subset, also a third subset used as a test database (which will also be referred to as “test set”).

In such a case, the first subset is used as the training set.

The third subset (test set) is used to calculate the precision and sensitivity of the prediction at different decision thresholds and to determine the aforesaid optimized threshold, based on the calculation of precision and sensitivity at different thresholds, shown above.

The second subset is used as a validation database (validation set) of the algorithm by setting the aforesaid optimized threshold as a threshold.

For example, an appropriate dataset of approximately 8,000 variants known to be benign or pathogenic is used as a training dataset.

According to an embodiment of the method, the aforesaid first type condition comprises a statistical condition and/or a known prior condition verifiable on clinical or clinical-statistical databases accessible by electronic computing means. Said second type condition comprises a patient-specific condition which is verifiable based on patient-specific information provided as input to the electronic computing means.

According to an embodiment of the method, the genomic data D are provided as input to electronic processing media in a standard VCF format, which is itself known.

The VCF format reports the list of variants found as a result of DNA sequencing of one or more patients. The VCF format is a standard format (https://samtools.github.io/hts-specs/VCFv4.3.pdf).

A VCF file is a text file, which contains “meta-information” in lines which start with “##”, a header which starts with the “#” character. The rows which list the variants contain tab-separated information.

According to an embodiment of the method the pathogenicity/benignity criteria comprise pathogenicity criteria, in turn divided into subsets associated with various respective levels of evidence, and benignity criteria, in turn divided into subsets associated with various respective levels of evidence.

According to an option of implementation, the pathogenicity/benignity criteria comprise criteria defined by known clinical standards and/or studies.

In particular, according to an implementation example, the pathogenicity/benignity criteria comprise criteria defined by ACMG/AMP.

In total, ACMG/AMP, in its current version, defines 28 criteria, most of which can be assessed automatically because they refer to information in accessible archives or databases, while others depend on the specific patient being assessed, and therefore must be provided as input to the model/algorithm of this method.

According to this implementation example, the pathogenicity/benignity criteria thus comprise one or more of the following criteria:
PVS1
PS1, PS2, PS3, PS4
PM1, PM2, PM3, PM4, PM5, PM6
PP1, PP2, PP3, PP4, PP5
BA1
BS1, BS2, BS3, BS4
BP1, BP2, BP3, BP4, BP5, BP6, BP7,
   The aforesaid criteria are defined as follows:
PVS1: Variant of the “null” type in a gene where it is known that the loss of function of the gene results in the onset of the disease;
PS1: The same amino acid change has previously been interpreted as pathogenic, regardless of the type of nucleotide change;
PS2: De novo variant confirmed in a patient with the disease and no family history (confirmed maternity and paternity);
PS3: In vivo or in vitro functional studies confirm a damaging effect of the variant on the gene or gene product;
PS4: The prevalence of the variant in individuals affected by the disease is significantly increased compared to the prevalence in controls;
PM1: Variant located in a mutational hot-spot and/or in a critical and well-established functional domain, without benign variants;
PM2: Variant absent in controls (or at a very low frequency if the disease is recessive) in Exome Sequencing Project, 1000 Genomes Project or Exome Aggregation Consortium;
PM3: For recessive diseases, the variant is found in trans with a pathogenic variant;
PM4: The protein length changes as a result of an in-frame deletion/insertion in a non-repeat region or stop-loss variants;
PM5: Novel missense change at an amino acid residue where a different missense change was previously determined to be pathogenic;
PM6: Presumed de novo variant, but without confirmation of paternity and maternity;
PP1: Co-segregation with disease in multiple affected family members in a gene known to cause the disease;
PP2: Missense variant in a gene which has a low rate of benign missense variants and in which missense-type variants cause the disease;
PP3: Multiple evidences from computational tools support a deleterious effect of the variant on the gene or gene product;
PP4: The patient's phenotype or family history is highly specific for the disease with a single genetic etiology;
PP5: A reliable source reports the variant as pathogenic, but the evidence is not available to the laboratory to perform an independent assessment;
BA1: The allele frequency of the variant is >5% in Exome Sequencing Project, 1000 Genomes Project, or Exome Aggregation Consortium;
BS1: The allele frequency is greater than that which would be expected for the disease;
BS2: Variant observed in a healthy adult for a recessive (homozygous), dominant (heterozygous) or X-linked (hemizygous) disease, with full penetrance at a young age;
BS3: In vivo or in vitro functional studies show no damaging effect of the variant on the gene or gene product;
BS4: lack of segregation in affected family members;
BP1: Missense variant in a gene for which primarily truncating variants are known to cause the disease;
BP2: Observed in trans with a pathogenic variant for a dominant gene/disease and with full or observed penetrance in cis with a pathogenic variant in any inheritance pattern;
BP3: In-frame deletion or insertion in a repetitive region without a known function;
BP4: Multiple evidences from computational tools support a non-deleterious effect of the variant on the gene or gene product;
BP5: Variant found in a case with an alternate molecular basis for the development of the disease;
BP6: A reliable source reports the variant as benign, but the evidence is not available to the laboratory to perform an independent assessment;
BP7: Synonymous (silent) variant for which the splicing prediction algorithms predict no impact on the splice sequence, nor the creation of a new splice site AND the nucleotide is highly conserved.

A person skilled in the art will readily understand that the present method is not limited to the use of the aforesaid criteria, but can also be applied using criteria derived from different standards (e.g.: Rivera-Muñoz E. A., Milko L. V., Harrison S. M., et al. “ClinGen Variant Curation Expert Panel experiences and standardized processes for disease and gene-level specification of the ACMG/AMP guidelines for sequence variant interpretation”. Human Mutation. 2018 November; 39(11):1614-1622. DOI: 10.1002/humu.23645), or, it may also be applied using standards which will be updated or developed in the future, or it may make use of additional criteria identified in research activities.

For example, according to an implementation option of the method, the pathogenicity/benignity criteria further comprise the following non-ACMG criterion:
BP8: The same amino acid change has previously been determined to be benign, regardless of the type of nucleotide change.

According to an implementation option, only a subset of criteria, selected based on the type of disease or condition considered, are used.

According to a different implementation option, all the aforesaid criteria are used.

According to an implementation option of the aforesaid embodiment of the method, the following criteria relate to a first type condition (statistical condition and/or a known prior condition): PVS1, PS1, PS3, PS4, PM1, PM2, PM4, PM5, PP2, PP3, PP5, BA1, BS1, BS3, BP1, BP3, BP4, BP7, BP8. The other criteria, i.e., PS2, PM3, PM6, PP1, PP4, BS2, BS4, BP2, BP5, relate to a second type condition (patient-specific condition).

With reference now to the “levels of evidence”, mentioned above, it is worth noting that, according to an embodiment of the method, such “levels of evidence” comprise levels of evidence associated with pathogenicity and levels of evidence associated with benignity.

According to an implementation option, the levels of evidence comprise levels defined by known clinical standards.

According to a particular implementation example, the levels of evidence comprise ACMG/AMP-defined levels of evidence.

According to an embodiment of the method, the levels of evidence comprise one or more of the following levels of evidence:
Pathogenicity: Very Strong;
Pathogenicity: Strong;
Pathogenicity: Moderate;
Pathogenicity: Supporting;
Benignity: Stand Alone;
Benignity: Very Strong;
Benignity: Supporting.

According to an implementation option, all of the above levels of evidence are used.

Thus, in this case, the criteria are attributed to one of the seven different levels of evidence in favor of whether the variant is pathogenic or not.

More specifically, the following associations apply in this example:
criterion PVS1 is associated with the level of evidence “Pathogenicity—Very Strong”;
criteria PS1, PS2, PS3, PS4 are associated with the level of evidence “Pathogenicity—Strong”;
criteria PM1, PM2, PM3, PM4, PM5, PM6 are associated with the level of evidence “Pathogenicity—Moderate”;
criteria PP1, PP2, PP3, PP4, PP5 are associated with the level of evidence “Pathogenicity—Supporting”;
criterion BA1 is associated with the level of evidence “Benignity—Stand Alone”;
criteria BS1, BS2, BS3, BS4 are associated with the level of evidence “Benignity—Very Strong”;
criteria BP1, BP2, BP3, BP4, BP5, BP6, BP7, BP8 are associated with the level of evidence “Benignity—Supporting”.

According to an embodiment of the method, the step of verifying (S1) that a variant meets each of a set of pre-selected criteria and counting of the criteria met by a variant is performed by a first software program or module or tool 1 (referred to hereafter as “first software tool 1” for the sake of brevity) configured to perform the aforementioned functions based on consultation of medical/clinical databases or archives and based on user-supplied input information.

As shown in FIG. 1, such first software tool 1 receives first input information B1, associated with the aforementioned “conditions of the first type,” and further receives second input information B2, associated with the aforementioned “conditions of the second type.”

According to an implementation option, the first input information B1 comes from databases or medical/clinical records that the first software tool 1 can query and/or consult.

According to an implementation option, the second input information B2 is provided by means of an electronic interface (computer keyboard, or touch screen or other) known in itself.

According to various implementation options, the aforementioned first software tool 1 configured to perform the aforementioned functions may comprise a tool from a set of tools, known in themselves, adapted to implement the chosen guidelines (e.g., ACMG/AMP).

These may comprise, for example:
Li Q., Wang K. “InterVar: Clinical Interpretation of Genetic Variants by the 2015 ACMG-AMP Guidelines”. Am. J. Hum. Genet. 2017 02; 100(2):267-80.
Ravichandran V., Shameer Z., Kernel Y., Walsh M., Cadoo K., Lipkin S., et al. “Toward automation of germline variant curation in clinical cancer genetics”. Genet. Med. 2019 September; 21(9):2116-25
Xavier A., Scott R. J., Talseth-Palmer B. A. “TAPES: A tool for assessment and prioritisation in exome studies”. PLOS Comput. Biol. 2019 Oct. 15; 15(10):e1007453.
Scott A. D., Huang K. L., Weerasinghe A., et al. “CharGer: clinical Characterization of Germline variants”. Bioinformatics. 2019; 35(5):865-867. doi:10.1093/bioinformatics/bty649.

According to another implementation option, the aforementioned first software tool 1 is a proprietary tool (“eVai”, https://evai.engenome.com), adapted to implement the chosen guidelines in an optimized manner. In particular, the eVai tool makes it possible to obtain the classification according to official ACMG/AMP guidelines specific for each disease which may be associated with a variant.

According to an embodiment of the method, the aforementioned step of preparing (S2) input information I for the trained algorithm A is performed by a second program or module or software tool 2 (also named “second software tool 2” hereafter for the sake of brevity).

According to various possible implementation options, such second software tool 2 may be either integrated in or independent from the first software program/tool 1.

According to an embodiment of the method, the aforesaid input information I for the trained algorithm A comprises, for each genomic variant, an indication of the number of pathogenicity/benignity criteria which are met by said genomic variant for each of the levels of evidence considered.

According to an implementation option, the aforesaid input information I for the trained algorithm A comprises one or more tables, in which:
each row is associated with a respective genomic variant,
each column is associated with a respective one of the following groups of criteria by level of evidence:

nPVS=PVS1

nPS=PS1+PS2+PS3+PS4

nPM=PM1+PM2+PM3+PM4+PM5+PM6

nPP=PP1+PP2+PP3+PP4+PP5

nBA=BA1

nBS=BS1+BS2+BS3+BS4

nBP=BP1+BP2+BP3+BP4+BP5+BP6+BP7+BP8
each cell contains a number obtained from the sum corresponding to the group of the respective column, wherein each criterion of the group is associated with 1 if the criterion is met by the genomic variant of the respective row, and is associated with 0 if the criterion is not met by the genomic variant of the respective row.

It is worth noting that the aforementioned input information I for trained algorithm A may indeed consist of a rule to derive the classification of variants into pathogenic or benign based on how well that variant meets the pathogenicity/benignity criteria.

A person skilled in the art can readily understand that the present method is not limited to only the rules illustrated above, in an embodiment of the method, but can be performed by adopting different rules.

Not only: the rules themselves (i.e., the input information for the trained machine learning algorithm) are definable, or “customizable”, by the user of the method, again through the normal electronic interfaces used to interact with the processor or computer running the method.

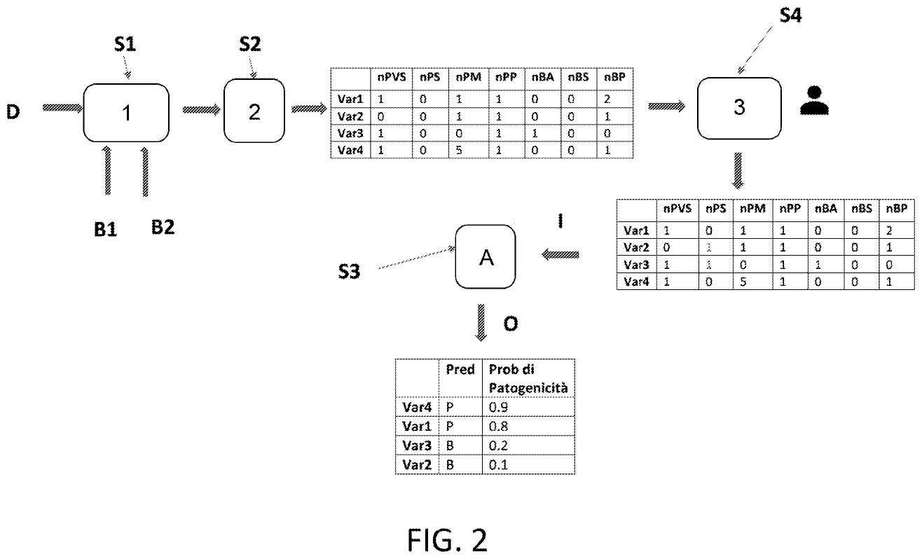

In particular, according to an embodiment shown in FIG. 2, the method comprises the further step of modifying (S4), by a user through an electronic interface 3 of said electronic computing means, the input information I for the trained algorithm A, before providing it as an input to the trained algorithm A.

In an implementation option, the user can decide to “enable” certain criteria (e.g., ACMG criteria), changing the number in the evidence levels accordingly.

According to other implementation options, such a number could also be modified “directly”, i.e., without going through the “standard” criteria listed above, and use specially defined criteria.

This function substantially makes it possible to “disconnect” from the standard guidelines, which apply in general, and apply other criteria, such as, for example, specific guidelines for given genes, which have been developed starting from the standard guidelines, and are available in literature.

Thus, the method described herein allows the user to apply any rule scheme for the interpretation of variants (typically, but not necessarily, maintaining the division according to levels of evidence defined by ACMG), thereby adding flexibility relative to different genes, and thus different diseases related thereto.

Indeed, the method can be used for the interpretation of variants in very rare Mendelian diseases (such as pediatric neurodevelopmental diseases), but also more complex diseases (such as cardiovascular or cancer predisposition).

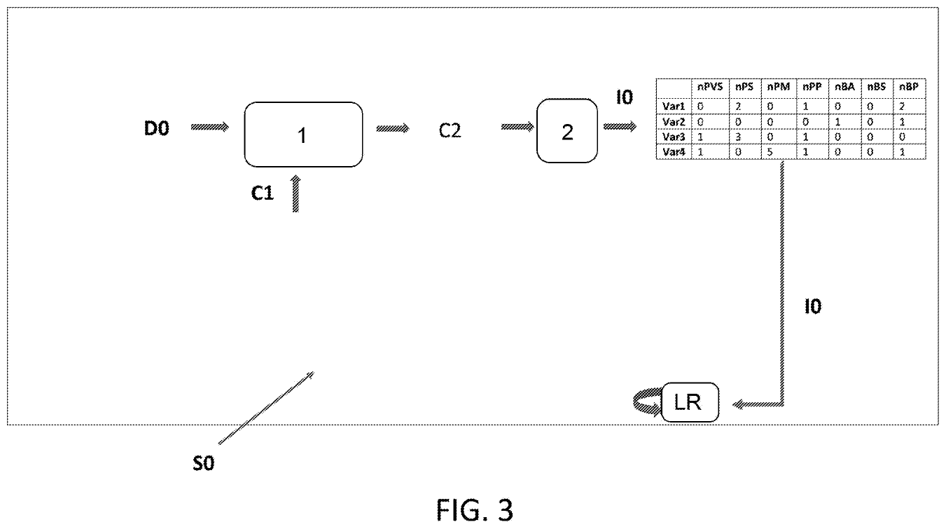

An option of implementation of the training step S0 of the method is described in detail below with reference to FIG. 3, by way of non-limiting example only.

The training genomic data D0 provided as input to the first software tool 1 are expressed as a VCF file (standard format) which contains the list of patient variants to be classified as pathogenic or benign (identified by position in the genome and amino acid change).

In the example considered, 3389 pathogenic variants and 5107 benign variants were obtained from the http://clinvitae.invita.com database to generate the training dataset. The resulting VCF file was provided as input to the first software tool 1.

Information C1 drawn from population databases (for example, ExAC, dbSNP, ESP) and/or archives of known variants (e.g., ClinVar) is further provided as input to software tool 1.

The first software tool 1 generates a piece of information C2 (for example, comprising an indication, for each variant, of the pathogenicity/benignity criteria, e.g., ACMG/AMP, which is met by the variant, and classification according to ACMG/AMP rules), which is provided as input to the second software tool 2.

The second software tool 2 performs a pre-processing which consists in aggregating and counting criteria by ACMG/AMP-defined levels of evidence, and in doing so prepares the input information I0 for the algorithm to be trained (which in this example is a logistic regression algorithm LR).

At this point, the training of the LR algorithm to be trained is performed in a standard manner on a training dataset (Clinvitae Training dataset).

Furthermore, in this example, also the step of choosing the optimal pathogenicity threshold on another test dataset (Clinvitae Test dataset) is performed as post-processing.

An example of classification of a variant will be reported hereinafter, purely by way of non-limiting example.

There is considered the variant located in chromosome 17, at position 41243451, where a patient carries nucleotide T instead of C (according to the reference genome). This variant is pathogenic for hereditary cancer according to the following study known from the literature: Mahamdallie S., Ruark E., Holt E., Poyastro-Pearson E., Renwick A., Strydom A., et al. “The ICR639 CPG NGS validation series: A resource to assess analytical sensitivity of cancer predisposition gene testing”. Wellcome Open Res. 2018; 3:68.

If one were to merely apply the ACMG/AMP guidelines, it can be noted that the variant verifies the following criteria:
PM1, because it is located in a hot-spot belonging to a protein domain and without benign variants in ClinVar (belonging to the “pathogenic moderate” level of evidence);
PM2, because it is not reported in population databases such as ESP, dbSNP, or ExAC;
PP3 because several in silico tools agree in predicting a damaging impact of the variant on the transcript (belonging to the “pathogenic supporting” level of evidence).

However, no ACMG/AMP final rule is verified, so according to the guidelines this variant is classified as uncertain.

Instead, by applying the trained machine learning model provided by this method, the variant has the following features:

| | nPVS | nPS | nPM | nPP | nBA | nBS | nBP |
|---|---|---|---|---|---|---|---|
| Var1 | 0 | 0 | 2 | 1 | 0 | 0 | 0 |

Using the aforementioned features as input for the trained algorithm, the regression model used predicts a probability of pathogenicity equal to 0.9931, which is greater than the optimized threshold of 0.86506 that the method itself established during another of its steps (previously illustrated) to be able to classify a pathogenic variant. As a result, the variant is classified as pathogenic.

As can be seen, the objects of the present invention as previously indicated are fully achieved by the method described above by virtue of the features shown above in detail.

Indeed, the method makes it possible to appropriately exploit the two approaches, respectively guidelines-based and data-driven, in a synergistic manner, by using the levels of evidence obtained from a tool which implements the guidelines considered (e.g., the ACMG/AMP guidelines) as features of a machine learning model trained on an appropriate training dataset.

Therefore, such a model makes it possible:
to improve classification/prediction performance, while relying on standard guidelines (e.g., ACMG/AMP) and thus allowing the user to understand the features of the model and to correctly check and interpret the operation and results of the method;
to provide a way to determine a priority among the variants, through the probability of classification provided as output by the model.

The latter is particularly important considering that thousands of variants can be analyzed in a patient. Consequently, it is important to report pathogenic variants as “first on the list” to facilitate the geneticist's task.

In light of the above, it is thus apparent that the method described here meets the need, increasingly felt in the considered technical field, to have tools for classifying genomic variants into benign or pathogenic which, on the one hand, relate to standard guidelines and, on the other hand, can classify as many variants into benign or pathogenic as possible (minimizing the number of “uncertain” variants) and ultimately improving the effectiveness and predictive accuracy.

A person skilled in the art may make changes and adaptations to the embodiments of the method described above or can replace elements with others which are functionally equivalent to satisfy contingent needs without departing from the scope of protection of the appended claims. All the features described above as belonging to a possible embodiment may be implemented independently of the other described embodiments.

1. A method for determining the pathogenicity/benignity of a genomic variant in connection with a given disease, comprising the steps of:
accessing genomic data comprising a list of the patient's genomic variants;
for each variant detected, verifying by a processor, whether or not the variant meets each of a plurality of predefined pathogenicity/benignity criteria,
wherein each pathogenicity/benignity criterion is a proposition, which can be true or false, related to the variant, in connection with a first type condition or a second type condition, and wherein at least one of said pathogenicity/benignity criteria refers to a first type condition, and at least another one of the pathogenicity/benignity criteria refers to a second type condition,
wherein said first type condition comprises a statistical condition and/or a previous known condition, and said second type condition comprises a condition specific of the patient,
wherein each pathogenicity/benignity criterion is associated with a level of evidence, indicative of a condition or level of pathogenicity or benignity;
preparing, by processing by the processor, input information for a trained algorithm,
wherein said input information comprises, for each variant and for each level of evidence, information representing the number of pathogenicity/benignity criteria associated with the level of evidence that are met by the variant;
processing said input information by the trained algorithm,
wherein said trained algorithm is an algorithm trained by artificial intelligence techniques and/or machine learning techniques,
wherein said algorithm is trained in a preliminary training step, based on a training dataset of known cases, providing the algorithm to be trained with said input information calculated for each of the known cases, and training the algorithm based on the knowledge of the pathogenicity/benignity of the respective known cases;
obtaining output information from the trained algorithm, said output information representing the pathogenicity/benignity of each of the genomic variants considered.

2. A method according to claim 1, wherein said output information comprises an estimated probability of pathogenicity of at least one genomic variant considered, or of a plurality of genomic variants among the genomic variants considered, or of all the genomic variants considered.

3. A method according to claim 2, wherein the output information further comprises, for each genomic variant, a binary result representing whether the genomic variant is pathogenic or benign, wherein said binary result is obtained by comparing a probability of pathogenicity estimated for the genomic variant with a respective threshold, associated with the genomic variant.

4. A method according to claim 3, wherein said respective threshold is an optimized threshold, common for all variants, and determined based on a pre-training.

5. A method according to claim 1, wherein said trained algorithm is a Logistic Regression algorithm, wherein said trained algorithm belongs to a group consisting of the following algorithms:
Decision Tree, Random Forest, Naive Bayes, Gradient Boosting, Support Vector Machine.

6. (canceled)

7. A method according to claim 1, comprising, before using said trained algorithms, a further preliminary training step, carried out based on two subsets of said training dataset containing data referring to known cases,
a first subset being used as a training database, and a second subset being used as a validation database.

8. A method according to claim 7, wherein said training dataset is divided into three subsets comprising, in addition to said first subset and second subset, also a third subset used as a test database,
and wherein the first subset is used as the training database, the third subset is used to calculate precision and sensitivity of the prediction at different decision thresholds and to determine said optimized threshold, based on said calculation of precision and sensitivity at different thresholds, and the second subset is used as a validation database of the algorithm by setting said optimized threshold as a threshold.

9. A method according to claim 1, wherein said first type condition comprises a statistical condition and/or a previous known condition which is verifiable on clinical or clinical-statistical databases accessible by the processor, and said second type condition comprises a specific condition of the patient, which is verifiable based on patient-specific input information provided to the processor.

10. A method according to claim 1, wherein the input genomic data are provided to the processor in a standard VCF format.

11. A method according to claim 1, wherein the pathogenicity/benignity criteria comprise pathogenicity criteria, the pathogenicity criteria being divided into subsets associated with various respective levels of evidence, and benignity criteria, the benignity criteria being divided into subsets associated with various respective levels of evidence, wherein the pathogenicity/benignity criteria comprise criteria defined by known clinical standards and/or studies, and/or wherein the pathogenicity/benignity criteria comprise criteria defined by ACMG.

12-13. (canceled)

14. A method according to claim 1, wherein the pathogenicity/benignity criteria comprise one or more of the following criteria:
PVS1
PS1, PS2, PS3, PS4
PM1, PM2, PM3, PM4, PM5, PM6
PP1, PP2, PP3, PP4, PP5
BA1
BS2, BS2, BS3, BS4
BP1, BP2, BP3, BP4, BP5, BP6, BP7,
wherein said criteria are defined as follows:
PVS1: Variant of the “null” type in a gene where the loss of function of the gene results in the onset of the disease is known;
PS1: The same amino acid change has previously been interpreted as pathogenic, regardless of the type of nucleotide change;
PS2: De novo variant confirmed in a patient with the disease and no family history (confirmed maternity and paternity);
PS3: In vivo or in vitro functional studies confirm a damaging effect of the variant on the gene or gene product;
PS4: The prevalence of the variant in individuals affected by the disease is significantly increased compared to the prevalence in controls;
PM1: Variant located in a mutational hot-spot and/or in a critical and well-established functional domain, without benign variants;
PM2: Variant absent in controls or at a very low frequency if the disease is recessive in Exome Sequencing Project, 1000 Genomes Project or Exome Aggregation Consortium;
PM3: For recessive diseases, the variant is found in trans with a pathogenic variant;
PM4: The protein length changes as a result of an in-frame deletion/insertion in a non-repeat region or stop-loss variants;
PM5: Novel missense change at an amino acid residue where a different missense change was previously determined to be pathogenic;
PM6: Presumed de novo variant, but without confirmation of paternity and maternity;
PP1: Co-segregation with disease in multiple affected family members in a gene known to cause the disease;
PP2: Missense variant in a gene which has a low rate of benign missense variants and in which missense-type variants cause the disease;
PP3: Multiple evidences from computational tools support a deleterious effect of the variant on the gene or gene product;
PP4: The patient's phenotype or family history is highly specific for the disease with a single genetic etiology;
PP5: A reliable source reports the variant as pathogenic, but the evidence is not available to the laboratory to perform an independent assessment;
BA1: The allele frequency of the variant is >5% in Exome Sequencing Project, 1000 Genomes Project, or Exome Aggregation Consortium;
BS1: The allele frequency is greater than that which would be expected for the disease;
BS2: Variant observed in a healthy adult for a recessive (homozygous), dominant (heterozygous) or X-linked (hemizygous) disease, with full penetrance at a young age;
BS3: In vivo or in vitro functional studies show no damaging effect of the variant on the gene or gene product;
BS4: lack of segregation in affected family members;
BP1: Missense variant in a gene for which primarily truncating variants are known to cause the disease;
BP2: Observed in trans with a pathogenic variant for a dominant gene/disease and with full or observed penetrance in cis with a pathogenic variant in any inheritance pattern;
BP3: In-frame deletion or insertion in a repetitive region without a known function;
BP4: Multiple evidence from computational tools support a non-deleterious effect of the variant on the gene or gene product;
BP5: Variant found in a case with an alternate molecular basis for the development of the disease;
BP6: A reliable source reports the variant as benign, but the evidence is not available to the laboratory to perform an independent assessment;
BP7: Synonymous (silent) variant for which the splicing prediction algorithms predict no impact on the splice sequence, nor the creation of a new splice site AND the nucleotide is highly conserved.

15. A method according to claim 14, wherein the pathogenicity/benignity criteria further comprise the following non-ACMG criterion:
BP8: The same amino acid change has previously been determined to be benign, regardless of the type of nucleotide change.

16. A method according to claim 14, wherein a subset of criteria is selected based on the type of illness or disease considered, wherein all of the criteria are used.

17. (canceled)

18. A method according to claim 14, wherein:
the following criteria relate to a first type condition, i.e., to a statistical condition and/or a previous known condition: PVS1, PS1, PS3, PS4, PM1, PM2, PM4, PM5, PP2, PP3, PP5, BA1, BS1, BS3, BP1, BP3, BP4, BP7, BP8;
and the following criteria relate to a second type condition, i.e., a condition specific of the patient: PS2, PM3, PM6, PP1, PP4, BS2, BS4, BP2, BP5.

19. A method according to claim 1, wherein the levels of evidence comprise levels of evidence associated with pathogenicity and levels of evidence associated with benignity, wherein the levels of evidence comprise levels defined by known clinical standards, and/or wherein the levels of evidence comprise levels of evidence defined by ACMG.

20-21. (canceled)

22. A method according to claim 1, wherein the levels of evidence comprise one or more of the following levels of evidence:
“Pathogenicity: Very Strong”;
“Pathogenicity: Strong”;
“Pathogenicity: Moderate”;
“Pathogenicity: Supporting”;
“Benignity: Stand Alone”;
“Benignity: Very Strong”;
“Benignity: Supporting”.

23. A method according to claim 22, wherein all of the above levels of evidence are used.

24. A method according to claim 14, wherein the following associations apply:
criterion PVS1 is associated with the level of evidence “Pathogenicity—Very Strong”;
criteria PS1, PS2, PS3, PS4 are associated with the level of evidence “Pathogenicity—Strong”;
criteria PM1, PM2, PM3, PM4, PM5, PM6 are associated with the level of evidence “Pathogenicity—Moderate”;
criteria PP1, PP2, PP3, PP4, PP5 are associated with the level of evidence “Pathogenicity—Supporting”;
criterion BA1 is associated with the level of evidence “Benignity—Stand Alone”;
criteria BS2, BS2, BS3, BS4 are associated with the level of evidence “Benignity—Very Strong”;
criteria BP1, BP2, BP3, BP4, BP5, BP6, BP7, BP8 are associated with the level of evidence “Benignity—Supporting”.

25. A method according to claim 1, wherein said input information for the trained algorithm comprises, for each genomic variant, an indication of the number of pathogenicity/benignity criteria that are met by said genomic variant for each of the levels of evidence considered, wherein said input information for the trained algorithm comprises one or more tables, wherein:
each row is associated with a respective genomic variant,
each column is associated with a respective one of the following groups of criteria by level of evidence:
nPVS=PVS1
nPS=PS1+PS2+PS3+PS4
nPM=PM1+PM2+PM3+PM4+PM5+PM6
nPP=PP1+PP2+PP3+PP4+PP5
nBA=BA1
nBS=BS2+BS2+BS3+BS4
nBP=BP1+BP2+BP3+BP4+BP5+BP6+BP7+BP8
each cell contains a number obtained from the sum corresponding to the group of the respective column, wherein each criterion of the group is associated with 1 if the criterion is met by the genomic variant of the respective row, and is associated with 0 if the criterion is not met by the genomic variant of the respective row.

26. (canceled)

27. A method according to claim 1, comprising the further step of:
modifying by a user through an electronic interface of said processor, the input information for the trained algorithm, before providing the input information as an input to the trained algorithm, wherein said modification step comprises activating one or more of said predefined pathogenicity/benignity criteria, and changing the number of the respective levels of evidence, or defining new criteria desired by the user and preparing the input information by inserting values related to said user-defined criteria.

28. (canceled)
